(19) United States
(12) Patent Application Publication
(10) Pub. No.: US 2025/0339596 A1
(43) Pub. Date: Nov. 6, 2025

(54) CONDUCTIVITY SENSOR

(71) Applicant: MOZARC MEDICAL US LLC, Minneapolis, MN (US)

(72) Inventors: Niccolo Chelli, Mirandola (IT); Giuliano Giganti, Mirandola (IT); Elisa Musella, Mirandola (IT); Riccardo Amadio, Mirandola (IT)

(21) Appl. No.: 19/199,084

(22) Filed: May 5, 2025

Related U.S. Application Data
(60) Provisional application No. 63/643,206, filed on May 6, 2024.

(51) Int. Cl. A61M 1/28; G01N 27/07; G01N 27/08; H01B 1/02; H01B 1/12

(52) U.S. Cl. CPC A61M 1/28; G01N 27/07; G01N 27/08; H01B 1/02; H01B 1/124; A61M 2205/0233; A61M 2205/025; A61M 2205/0283

(57) ABSTRACT

A system for performing dialysis treatment includes a water purification system configured to purify a fluid intended for dialysis, the system including a conductivity sensor including a sensor body, fluid inlet, fluid outlet, first electrode, and second electrode. The sensor body enclosing a fluid flow path between the fluid inlet and fluid outlet. The first electrode is disposed in the fluid flow path between the fluid inlet and the fluid outlet. the first electrode includes a metallic substrate, and a coating covering at least a portion of the metallic substrate. The second electrode is located in the fluid flow path between the fluid inlet and the fluid outlet. The coating can be a conductive polymer. The conductive polymer can be polypyrrole. The polypyrrole can be doped with sodium dodecyl sulfate. The polypyrrole can be doped with graphene. The conductive polymer includes poly (3,4-ethylenedioxythiophene) polystyrene sulfonate.

### CROSS REFERENCE

This application claims the benefit of and priority to U.S. Provisional Patent Application No. 63/643,206 filed May 6, 2024, the entire content of each of which is incorporated by reference herein.

### FIELD

This disclosure relates generally to a conductivity sensor. More particularly, this disclosure relates to a conductivity sensor configured for use in dialysis systems.

### BACKGROUND

Dialysis systems can be used to treat patients with kidney disorders. There are a number of dialysis systems in use in the health care industry. Purified water that is specifically controlled for the dialysis systems is used in these dialysis systems for treatment of the patients.

### SUMMARY

In some embodiments, a dialysis system includes a purification system configured to purify a fluid intended for dialysis, and a conductivity sensor including a sensor body. In some embodiments, the sensor body enclosing a fluid flow path. In some embodiments, the sensor body has a fluid inlet and a fluid outlet. In some embodiments, the conductivity sensor includes a first electrode. In some embodiments, the first electrode is disposed in the fluid flow path between the fluid inlet and the fluid outlet. In some embodiments, the first electrode includes a metallic substrate and a coating covering at least a portion of the metallic substrate. In some embodiments, the conductivity sensor includes a second electrode. In some embodiments, the second electrode is located in the fluid flow path between the fluid inlet and the fluid outlet.

In some embodiments, the coating includes a conductive polymer.

In some embodiments, the conductive polymer includes polypyrrole.

In some embodiments, the polypyrrole is doped with sodium dodecyl sulfate.

In some embodiments, the polypyrrole is doped with graphene.

In some embodiments, the polymer includes poly (3,4-ethylenedioxythiophene) polystyrene sulfonate.

In some embodiments, a conductivity sensor includes a sensor body. In some embodiments, the sensor body enclosing a fluid flow path. In some embodiments, the sensor body has a fluid inlet and a fluid outlet. In some embodiments, the conductivity sensor includes a first electrode. In some embodiments, the first electrode is disposed in the fluid flow path between the fluid inlet and the fluid outlet. In some embodiments, the first electrode includes a metallic substrate and a coating covering at least a portion of the metallic substrate. In some embodiments, the conductivity sensor includes a second electrode. In some embodiments, the second electrode is located in the fluid flow path between the fluid inlet and the fluid outlet.

In some embodiments, the metallic substrate includes stainless steel.

In some embodiments, the metallic substrate includes aluminum.

In some embodiments, the coating includes a conductive polymer.

In some embodiments, the conductive polymer includes polypyrrole.

In some embodiments, the polypyrrole is doped with sodium dodecyl sulfate.

In some embodiments, the polypyrrole is doped with sodium p-toluenesulfonate.

In some embodiments, the polypyrrole is doped with graphene.

In some embodiments, the conductive polymer includes poly (3,4-ethylenedioxythiophene) polystyrene sulfonate.

In some embodiments, the coating includes one or more additives.

In some embodiments, the one or more additives are configured to improve a corrosion resistance, improve an electrical conductivity, or combinations thereof.

In some embodiments, the first electrode is a first cylinder, and the second electrode is a second cylinder. In some embodiments, the first cylinder and the second cylinder are disposed so that longitudinal axes thereof are parallel.

In some embodiments, a conductivity sensor includes a sensor body, the sensor body enclosing a fluid flow path. In some embodiments, the sensor body has a fluid inlet and a fluid outlet. In some embodiments, the conductivity sensor includes a first electrode. In some embodiments, the first electrode is disposed in the fluid flow path between the fluid inlet and the fluid outlet. In some embodiments, the first electrode includes a metallic substrate and a coating covering at least a portion of the metallic substrate. In some embodiments, the conductivity sensor includes a second electrode. In some embodiments, the second electrode is located in the fluid flow path between the fluid inlet and the fluid outlet. In some embodiments, the first electrode is a first plate, and the second electrode is a second plate. In some embodiments, the first plate and the second plate are disposed so that major surfaces thereof are parallel.

In some embodiments, a conductivity sensor includes a sensor body, the sensor body enclosing a fluid flow path. In some embodiments, the sensor body has a fluid inlet and a fluid outlet. In some embodiments, the conductivity sensor includes a first electrode. In some embodiments, the first electrode is disposed in the fluid flow path between the fluid inlet and the fluid outlet. In some embodiments, the first electrode includes a metallic substrate and a coating covering at least a portion of the metallic substrate. In some embodiments, the conductivity sensor includes a second electrode. In some embodiments, the second electrode is located in the fluid flow path between the fluid inlet and the fluid outlet wherein the first electrode and the second electrode include arrays of interdigitated pins.

In some embodiments, a conductivity sensor includes a sensor body, the sensor body enclosing a fluid flow path. In some embodiments, the sensor body has a fluid inlet and a fluid outlet. In some embodiments, the conductivity sensor includes a first electrode. In some embodiments, the first electrode is disposed in the fluid flow path between the fluid inlet and the fluid outlet. In some embodiments, the first electrode includes a metallic substrate and a coating covering at least a portion of the metallic substrate. In some embodiments, the conductivity sensor includes a second electrode. In some embodiments, the conductivity sensor includes a second electrode. In some embodiments, the second electrode is located in the fluid flow path between the fluid inlet and the fluid outlet. In some embodiments, the first electrode is a first cylinder, and the second electrode is a second cylinder. In some embodiments, the first cylinder and the second cylinder are colinear. In some embodiments, at least a portion of the first cylinder is disposed within an interior of the second cylinder.

### BRIEF DESCRIPTION OF THE DRAWINGS

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

FIG. 1 is a schematic diagram of a dialysis system, according to some embodiments.

FIG. 2 is a partially exposed side view of electrodes, according to some embodiments.

FIG. 3 is a partially exposed perspective view of electrodes, according to some embodiments.

FIG. 4 is a partially exposed side view of electrodes, according to some embodiments.

FIG. 5 is a perspective view of electrodes, according to some embodiments.

FIG. 6 is a partially exposed side view of the electrodes in FIG. 5, according to some embodiments.

FIG. 7 is a top view of the electrodes in FIG. 5, according to some embodiments.

FIG. 8 is a perspective view of the conductivity sensor, according to some embodiments.

Like reference numbers represent the same or similar parts throughout.

### DETAILED DESCRIPTION

Dialysis systems such as, but not limited to, hemodialysis, hemofiltration, hemodiafiltration, and peritoneal dialysis, can utilize a purified water source for the dialysis treatments. In some embodiments, the dialysis systems can utilize a water for injection (WFI), which is a form of sterile water to deliver medications or drugs to patients. The systems disclosed are designed to reduce risk of contaminants and to ensure an appropriate composition of the purified or sterile water.

Embodiments of this disclosure are directed to systems, devices, and apparatuses for conductivity sensors capable of retaining their one or more properties throughout a lifetime of the sensor. In some embodiments, the conductivity sensor can be capable of retaining their one or more properties when utilized in chemically aggressive environments. The one or more properties can include, but is not limited to, electrical conductivity and corrosion resistance. In some embodiments, the chemically aggressive environments can include certain fluids. In some embodiments, the chemically aggressive environments can include WFI to deliver medications or drugs to a patient, as a cleaning agent, or suitable combinations thereof.

In some embodiments, the conductivity sensor includes one or more electrodes. The one or more electrodes can include polymers. That is, the one or more electrodes can be manufactured using polymers. In some embodiments, the electrodes can be formed during a manufacture stage by electrochemically depositing one or more layers of polymers on a metallic substrate starting from a conductive solution. The result is an electrode capable of resisting corrosion when utilized in chemically aggressive environments. The resulting electrode is also electrically conductive to measure a concentration of electrically charged particles in a treatment fluid. The resulting electrode is also more cost effective to manufacture. In some embodiments, the electrode can include poor metallic substrates that are typically lower in cost as compared with other metallic substrates, thereby reducing the cost to manufacture the electrodes, the sensors, or combinations thereof. In some embodiments, the metallic substrate of the electrodes can be formed using one or more metallic materials without including certain metallic materials such as titanium, platinum, silver, copper, bronze, and gold, which have a higher cost. The resulting electrode is capable of measuring electrical conductivity in fluids without including titanium, platinum, silver, copper, bronze, gold, or any combinations thereof. The resulting electrodes can also be manufactured without treatments such as, but not limited to, chrome, nickel, and gold plating. In some embodiments, not using such treatments can eliminate a need for certain post processing treatments and reduce manufacturing time.

Among those benefits and improvements that have been disclosed, other objects and advantages of this disclosure will become apparent from the following description taken in conjunction with the accompanying figures. Detailed embodiments of the present disclosure are disclosed herein. It is to be understood that the disclosed embodiments are merely illustrative of the disclosure that may be embodied in various forms. In addition, each of the examples given regarding the various embodiments of the disclosure which are intended to be illustrative, and not restrictive.

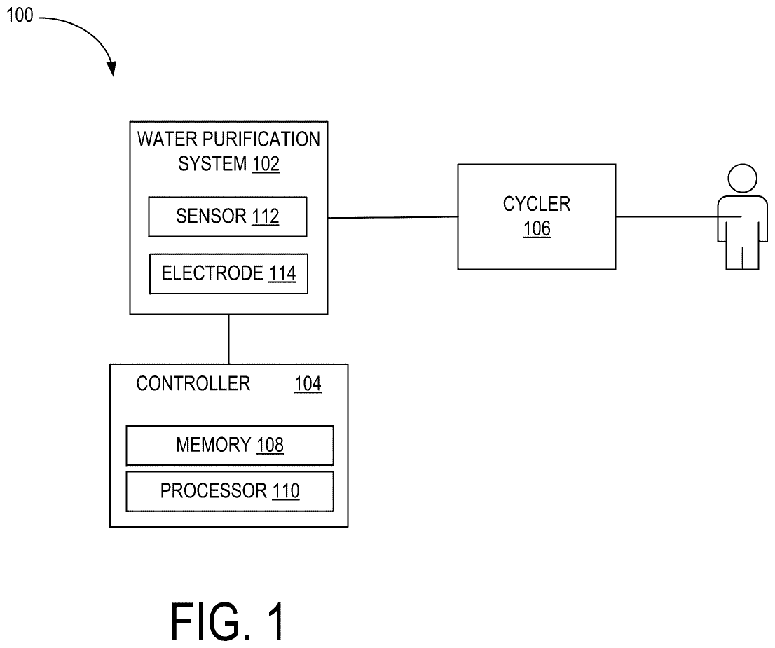

FIG. 1 is a schematic diagram of a dialysis system 100, according to some embodiments. In some embodiments, the dialysis system 100 can be representative of a peritoneal dialysis system including point of use dialysis fluid production. Peritoneal dialysis systems are one example of a dialysis system. It is to be appreciated that the systems and methods described in this disclosure can be applied to other dialysis systems such as, but not limited to, hemodialysis, hemofiltration, hemodiafiltration, or the like.

The illustrated embodiment includes a water purification system 102. The water purification system 102 includes a conductivity sensor 112 and one or more electrodes 114. In some embodiments, the conductivity sensor 112 includes the one or more electrodes 114. The conductivity sensor 112 can be in fluid communication with the fluid in the water purification system 102. The fluid in the water purification system 102, or at least a portion thereof, can flow through a fluid flow path of the conductivity sensor 112. The one or more electrodes 114 can be located in the fluid flow path of the conductivity sensor 112, as will be further described herein.

Although not shown in FIG. 1, the water purification system 102 can include one or more systems therein such as, but not limited to, a pretreatment system, a treatment system, a distribution system, or combinations thereof. In addition, in some embodiments, the conductivity sensor 112, the one or more electrodes 114, or suitable combinations thereof can be located within one or more of these systems in the water purification system 102.

A controller 104 is configured to be in electronic communication with the water purification system 102 to send and receive communications relating to sensed parameters, control of valves, or the like. The water purification system 102 can be fluidly connected to a cycler 106.

The cycler 106 can be configured to control the phases (e.g., delivery, dwell, and drain) of therapy using dialysis fluid (e.g., that is prepared using the purified water from the water purification system 102). The cycler 106 can be fluidly connected with a patient to perform the dialysis treatments. The cycler 106 can be configured to inject the dialysis fluid into the patient (e.g., delivery phase) and drain the dialysis fluid when the treatment is complete (e.g., drain phase after completion of a dwell phase). The cycler 106 can be in electronic communication with the controller 104 to accomplish the necessary treatments for the patient.

In some embodiments, another domain in the water purification system 102 may prepare the fresh dialysis fluid using purified water. For example, the water purification system 102 may include a preparator for mixing the fresh dialysis fluid using the WFI. The preparator can be in electronic communication with the controller 104 to accomplish the necessary treatments for the patient. In some embodiments, the preparator can be in electronic communication with the cycler 106 to accomplish the necessary treatments for the patient. It is to be appreciated that the cycler 106 can include one or more additional features such as, but not limited to, a user interface configured to receive user inputs, display outputs for the user, or any combination thereof.

The controller 104 can be in wired or wireless communication with the water purification system 102. The controller 104 can include a memory 108 and at least one processor 110. It is to be appreciated that the controller 104 can include one or more additional features such as, but not limited to, a display with a user interface configured to receive user inputs, display outputs for the user, or any combination thereof. In some embodiments, a separate user input can also be included so the user can interact with the dialysis system 100.

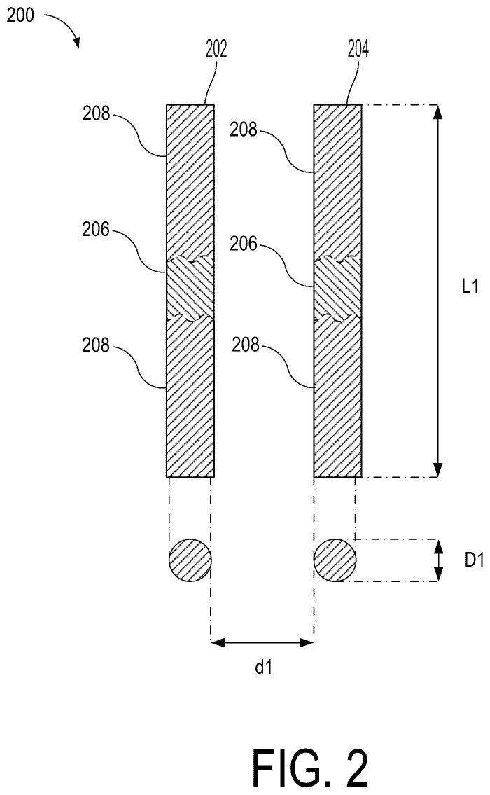

According to some embodiments, the conductivity sensor 112 can include one or more electrodes 114. According to some embodiments, the one or more electrodes 114 can be electrodes 200, 300, 400, 500, as shown in FIGS. 2-7, and as will be further described herein.

The conductivity sensor 112 is designed to measure conductivities within a conductivity sensing range of 0.5 to 150 μS/cm (micro-Siemens per centimeter), or any range or subrange therebetween. In some embodiments, the conductivity sensor 112 can be designed to have an accuracy of +/−0.1 μS/cm. In some embodiments, the conductivity sensor 112 can be configured to have an accuracy of +/−1 μS/cm. In some embodiments, the conductivity sensor 112 can be configured to have an accuracy of +/−0.1 μS/cm, +/−0.2 μS/cm, +/−0.3 μS/cm +/−, +/−0.4 μS/cm, +/−0.5 μS/cm, +/−1 μS/cm, +/−2 μS/cm, and +/−5 μS/cm, depending on various factors such as, for example, application, fluid properties, conductivity sensing range, and the like.

In some embodiments, the conductivity sensor 112 is configured to measure conductivities within a conductivity sensing range of 0.5 to 150 μS/cm, 0.5 to 125 μS/cm, 0.5 to 100 μS/cm, 0.5 to 75 μS/cm, 0.5 to 50 μS/cm, 0.5 to 25 μS/cm, 0.5 to 20 μS/cm, 0.5 to 15 μS/cm, 0.5 to 10 μS/cm, 0.5 to 5 μS/cm, 0.5 to 3 μS/cm, 0.5 to 2 μS/cm, 0.5 to 1 μS/cm, 1 to 150 μS/cm, 1 to 125 μS/cm, 1 to 100 μS/cm, 1 to 75 μS/cm, 1 to 50 μS/cm, 1 to 25 μS/cm, 1 to 20 μS/cm, 1 to 15 μS/cm, 1 to 10 μS/cm, 1 to 5 μS/cm, 1 to 3 μS/cm, 1 to 2 μS/cm, 5 to 150 μS/cm, 5 to 125 μS/cm, 5 to 100 μS/cm, 5 to 75 μS/cm, 5 to 50 μS/cm, 5 to 25 μS/cm, 5 to 20 μS/cm, 5 to 15 μS/cm, 5 to 10 μS/cm, 10 to 150 μS/cm, 10 to 125 μS/cm, 10 to 100 μS/cm, 10 to 75 μS/cm, 10 to 50 μS/cm, 10 to 25 μS/cm, 10 to 20 μS/cm, 10 to 15 μS/cm, 15 to 150 μS/cm, 15 to 125 μS/cm, 15 to 100 μS/cm, 15 to 75 μS/cm, 15 to 50 μS/cm, 15 to 25 μS/cm, 15 to 20 μS/cm, 25 to 150 μS/cm, 25 to 125 μS/cm, 25 to 100 μS/cm, 25 to 75 μS/cm, 25 to 50 μS/cm, 50 to 150 μS/cm, 50 to 125 μS/cm, 50 to 100 μS/cm, 50 to 75 μS/cm, and other ranges.

In a non-limiting example, the conductivity sensor 112 is configured to measure conductivities within a conductivity sensing range of 0.5 to 5 μS/cm with an accuracy of +/−0.1 μS/cm. In another non-limiting example, the conductivity sensor 112 is configured to measure conductivities within a conductivity sensing range of 0.5 to 10 μS/cm with an accuracy of +/−0.1 μS/cm. In yet another non-limiting example, the conductivity sensor 112 is configured to measure conductivities within a conductivity sensing range of 5 to 100 μS/cm with an accuracy of +/−1 μS/cm. In another example, the conductivity sensor 112 is configured to measure conductivities within a conductivity sensing range of 5 to 50 μS/cm with an accuracy of +/−1 μS/cm.

FIG. 2 is a partially exposed side view of electrodes 200, according to some embodiments.

In some embodiments, the electrodes 200 can be electrode 202 and electrode 204. Referring to FIG. 2, in some embodiments, each of the electrodes 200 can have a length (L1) and a diameter (D1). In some embodiments, the electrodes 200 can include electrode 202 having a length (L1) and a diameter (D1). In some embodiments, the electrodes 200 can include electrode 204 having a length (L1) and a diameter (D1). In addition, in some embodiments, the electrodes 200 can be spaced a distance (d1) apart. It is to be appreciated by those having ordinary skill in the art that the dimensions of the electrodes 200 are not intended to be limiting and may include different dimensions depending on the application. For example, the dimensions of the electrodes 200 may be dependent on the size of the conductivity sensor 112 in the water purification system 102.

In some embodiments, the electrode 202 can have a cylindrical shape. That is, the electrode 202 can be a cylinder. In some embodiments, the electrode 204 can have a cylindrical shape. That is, the electrode 204 can be a cylinder. In some embodiments, the electrode 202 and the electrode 204 can be disposed relative each other so that longitudinal axes thereof are parallel. In some embodiments, the electrode 202 and the electrode 204 can be disposed relative each other so that the longitudinal axes thereof are substantially parallel. For example, the longitudinal axis of the electrode 202 can be substantially parallel to a longitudinal axis of the electrode 204 so that a distance between a first end of electrode 202 and a corresponding first end of electrode 204 being the same or similar to a distance between a second end of electrode 202 and a corresponding second end of electrode 204.

In some embodiments, the longitudinal axes of the electrode 202 and the electrode 204 can be angularly offset relative each other. In some embodiments, the longitudinal axis of the electrode 202 can be angularly offset relative the longitudinal axis of the electrode 204 by 0° to 30°, or any range or subrange thereof. In some embodiments, the longitudinal axis of the electrode 202 can be angularly offset from the longitudinal axis of the electrode 204 by 0° to 1°, 0° to 2°, 0° to 5°, 0° to 10, 0° to 15°, 0° to 20°, 0° to 25°, or 0° to 30°. For example, the longitudinal axis of the electrode 202 can be angularly offset from the longitudinal axis of the electrode 204 by 3°. In another example, the longitudinal axis of the electrode 202 can be angularly offset from the longitudinal axis of the electrode 204 by 11°.

The electrodes 200 are configured to contact the fluid in the water purification system 102 such as, for example, WFI, to enable the conductivity sensor 112 to measure a concentration of electrically charged particles (e.g., ions) in the fluid so as to enable the controller 104 to ensure proper ion concentration in the fluid for treatment purposes. In some embodiments, if the concentration of electrically charged particles in the water is higher than desired, the conductivity will also be higher than expected. As a result, readings from the conductivity sensor 112 can be used to infer whether the one or more systems in the water purification system 102 are properly functioning at controlling the conductivity of the fluid. For example, higher than expected readings can indicate that a reverse osmosis membrane (not shown) in the water purification system 102 is not operating as expected. Based on the conductivity readings from the conductivity sensor 112, the controller 104 may also control an operation of the water purification system 102 so as to, for example, drain the water and prevent water from continuing through the water purification system 102 by controlling an operation of one or more respective valves (not shown) in the water purification system 102 in cases in which the sensed values are outside of a specified conductivity requirement.

The electrodes 200 can include a metallic substrate 206. In some embodiments, the electrode 202 can include a metallic substrate 206. In some embodiments, the electrode 204 can include a metallic substrate 206. In some embodiments, the electrode 202 and the electrode 204 can include the metallic substrate 206.

In some embodiments, the metallic substrate 206 includes stainless steel. In other embodiments, the metallic substrate 206 can be composed substantially of stainless steel. In other embodiments, the metallic substrate 206 can be composed essentially of stainless steel. In some embodiments, the metallic substrate 206 includes aluminum. In other embodiments, the metallic substrate 206 can be composed substantially of aluminum. In other embodiments, the metallic substrate 206 can be composed essentially of aluminum. In some embodiments, the metallic substrate 206 can include stainless steel, aluminum, or combinations thereof.

The electrodes 200 can include a coating 208. In some embodiments, the coating 208 can cover at least a portion of the metallic substrate 206. In some embodiments, the electrode 202 can include the coating 208 covering at least a portion of the metallic substrate 206. In some embodiments, the electrode 204 can include the coating 208 covering at least a portion of the metallic substrate 206. In some embodiments, the coating 208 can cover the portion of the metallic substrate 206 that is in contact with the fluid in the fluid path. In some embodiments, the coating 208 can substantially cover the metallic substrate 206.

In some embodiments, the coating 208 can be a conductive polymer. In some embodiments, the conductive polymer includes polypyrrole. In some embodiments, the polypyrrole can be present at a concentration of 0.5 to 1.5 mol/L. In other embodiments, the conductive polymer can be composed substantially of polypyrrole. In other embodiments, the conductive polymer can be composed essentially of polypyrrole. In some embodiments, the conductive polymer includes poly (3,4-ethylenedioxythiophene) polystyrene sulfonate. In some embodiments, the conductive polymer includes polypyrrole, poly (3,4-ethylenedioxythiophene) polystyrene sulfonate, or any combinations thereof.

In some embodiments, the conductive polymer includes a doping agent. In some embodiments, the polypyrrole can be doped with sodium dodecyl sulfate. In some embodiments, the sodium dodecyl sulfate can be present at a concentration of 0.05 mol/L to 0.5 mol/L. In some embodiments, the polypyrrole can be doped with graphene. In some embodiments, the polypyrrole can be doped with sodium p-toluenesulfonate. In some embodiments, the p-toluenesulfonate can be present at a concentration of 0.05 mol/L to 0.5 mol/L. In some embodiments, the polypyrrole can be doped with sodium dodecyl sulfate, graphene, sodium p-toluenesulfonate, or any combinations thereof.

In some embodiments, the coating 208 includes one or more additives. In some embodiments, the one or more additives are configured to improve a corrosion resistance, improve an electrical conductivity, or combinations thereof. For example, the coating 208 can include an additive for improving a corrosion resistance properties of the electrodes 200. In another example, the coating 208 can include an additive for improving an electrical conductivity of the electrodes 200.

For example, in some embodiments, the coating 208 is a conductive polymer including polypyrrole doped with sodium dodecyl sulfate and can include one or more additives. In some embodiments, the coating can be a conductive polymer including polypyrrole, poly (3,4-ethylenedioxythiophene) polystyrene sulfonate, sodium dodecyl sulfate, graphene, additives, or any combinations thereof.

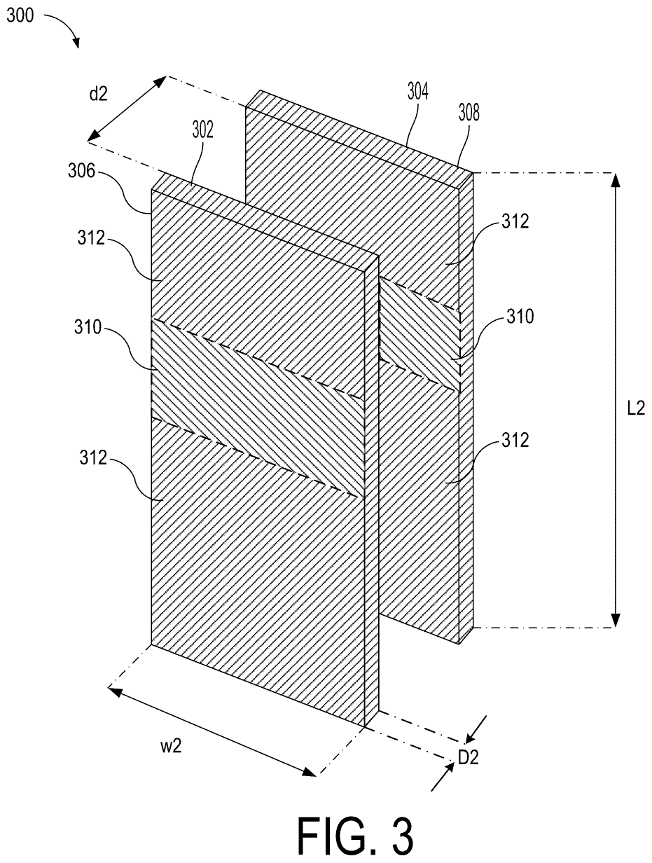

FIG. 3 is a partially exposed perspective view of electrodes 300, according to some embodiments.

In some embodiments, the one or more electrodes 300 can be electrode 302 and electrode 304. In some embodiments, the one or more electrodes 300 can include electrode 302 and electrode 304. The electrode 302 and the electrode 304 can each have a length (L2), a width (w2), and a thickness (D2). In addition, the electrode 302 and the electrode 304 can be spaced apart from each other by a distance (d2).

In some embodiments, the electrode 302 can be a plate 306. In some embodiments, the electrode 304 can be a plate 308. In some embodiments, the electrode 302 can be plate 306 and electrode 304 can be plate 308. In some embodiments, the electrode 302 can be disposed relative electrode 304 in the conductivity sensor 112 so that major surfaces thereof are parallel to each other. That is, in some embodiments, the electrode 302 and electrode 304 can extend along respective directions so that major surfaces thereof are parallel.

In some embodiments, the electrode 302 and electrode 304 can be disposed in the fluid flow path between the fluid inlet and the fluid outlet of the conductivity sensor 112.

In some embodiments, the electrode 302 can include the metallic substrate 310. In some embodiments, the electrode 302 can also include the coating 312. In some embodiments, the coating 312 can cover at least a portion of the metallic substrate 310. In some embodiments, the coating 312 can cover the portion of the metallic substrate 310 that is in contact with the fluid in the fluid path. In other embodiments, the coating 312 can substantially cover the metallic substrate 310.

In some embodiments, the electrode 304 can include the metallic substrate 310. In some embodiments, the electrode 304 can also include the coating 312. In some embodiments, the coating 312 can cover at least a portion of the metallic substrate 310. In some embodiments, the coating 312 can cover the portion of the metallic substrate 310 that is in contact with the fluid in the fluid path. In other embodiments, the coating 312 can substantially cover the metallic substrate 310.

In some embodiments, the metallic substrate 310 includes stainless steel. In other embodiments, the metallic substrate 310 can be composed substantially of stainless steel. In other embodiments, the metallic substrate 310 can be composed essentially of stainless steel. In some embodiments, the metallic substrate 310 includes aluminum. In other embodiments, the metallic substrate 310 can be composed substantially of aluminum. In other embodiments, the metallic substrate 310 can be composed essentially of aluminum. In some embodiments, the metallic substrate 310 can include stainless steel, aluminum, or combinations thereof.

In some embodiments, the coating 312 can be a conductive polymer. In some embodiments, the conductive polymer includes polypyrrole. In other embodiments, the conductive polymer can be composed substantially of polypyrrole. In other embodiments, the conductive polymer can be composed essentially of polypyrrole. In some embodiments, the conductive polymer includes poly (3,4- ethylenedioxythiophene) polystyrene sulfonate. In some embodiments, the conductive polymer includes polypyrrole, poly (3,4-ethylenedioxythiophene) polystyrene sulfonate, or any combinations thereof.

In some embodiments, the conductive polymer includes a doping agent. In some embodiments, the polypyrrole can be doped with sodium dodecyl sulfate. In some embodiments, the polypyrrole can be doped with graphene. In some embodiments, the polypyrrole can be doped with sodium p-toluenesulfonate. In some embodiments, the polypyrrole can be doped with sodium dodecyl sulfate, graphene, sodium p-toluenesulfonate, or any combinations thereof.

In some embodiments, the coating 312 includes one or more additives. In some embodiments, the one or more additives are configured to improve a corrosion resistance, improve an electrical conductivity, or combinations thereof. For example, the coating 312 can include an additive for improving a corrosion resistance properties of the electrodes 300. In another example, the coating 312 can include an additive for improving an electrical conductivity of the electrodes 300.

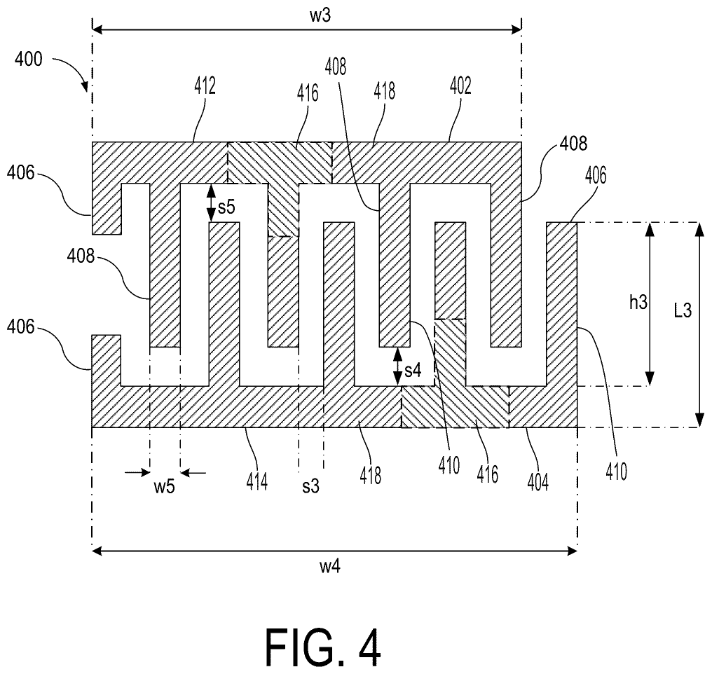

FIG. 4 is a partially exposed side view of electrodes 400, according to some embodiments.

In some embodiments, the electrodes 400 can be electrode 402 and electrode 404. In some embodiments, the one or more electrodes 400 can include electrode 402 and electrode 404. In some embodiments, the electrode 402 and the electrode 404 can include one or more pins 406.

In some embodiments, the electrode 402 can include an array of pins 408. In some embodiments, the electrode 404 can include an array of pins 410. In some embodiments, in the conductivity sensor 112, the electrode 402 and the electrode 404 can be positioned relative each other so that the array of pins 408 located on electrode 402 are interdigitated with the respective array of pins 410 located on electrode 404. That is, in some embodiments, the pins 408 of electrode 402 can be arranged so as to be interspersed with the pins 410 of electrode 404 in an alternating arrangement. In some embodiments, the electrode 402 can be arranged relative the electrode 404 so that one or more pins 408 of the array of pins 408 of electrode 402 are interdigitated with one or more pins 410 of the array of pins 410 of electrode 404.

In some embodiments, the electrode 402 can include base member 412. In some embodiments, the array of pins 408 can extend from the base member 412. In some embodiments, the array of pins 408 can extend from a same side of the base member 412. In some embodiments, the array of pins 408 can extend from the base member 412 in a parallel direction relative the other pins in the array of pins 408.

In some embodiments, the electrode 404 can include base member 414. In some embodiments, the array of pins 410 can extend from the base member 414. In some embodiments, the array of pins 410 can extend from a same side of the base member 414. In some embodiments, the array of pins 410 can extend from the base member 414 in a parallel direction relative the other pins in the array of pins 410.

In some embodiments, the electrode 402 and electrode 404 can each have a length (L3). In some embodiments, the base member 412 and the base member 414 can have a width (w3) and a width (w4), respectively. In addition, in some embodiments, the pins 408 and pins 410 can each have a height (h3) and a width (w5). In some embodiments, when the electrode 402 is arranged relative the electrode 404 in the conductivity sensor 112 so that the array of pins 408 are interdigitated between the array of pins 410, the pins 408 and the pins 410 can be spaced apart by a distance (s3). In some embodiments, the space between each of the pins 408 and the opposite base member 414 can be set a distance (s4). In some embodiments, the space between each of the pins 410 and the opposite base member 412 can be set a distance (s5)

In some embodiments, the electrode 402 and electrode 404 can be disposed in the fluid flow path between the fluid inlet and the fluid outlet of the conductivity sensor 112.

In some embodiments, the electrode 402 can include the metallic substrate 416. In some embodiments, the electrode 402 can also include the coating 418. In some embodiments, the coating 418 can cover at least a portion of the metallic substrate 416. In some embodiments, the coating 418 can cover the portion of the metallic substrate 416 that is in contact with the fluid in the fluid path. In other embodiments, the coating 418 can substantially cover the metallic substrate 416.

In some embodiments, the electrode 404 can include the metallic substrate 416. In some embodiments, the electrode 404 can also include the coating 418. In some embodiments, the coating 418 can cover at least a portion of the metallic substrate 416. In some embodiments, the coating 418 can cover the portion of the metallic substrate 416 that is in contact with the fluid in the fluid path. In other embodiments, the coating 418 can substantially cover the metallic substrate 416.

In some embodiments, the metallic substrate 416 includes stainless steel. In other embodiments, the metallic substrate 416 can be composed substantially of stainless steel. In other embodiments, the metallic substrate 416 can be composed essentially of stainless steel. In some embodiments, the metallic substrate 416 includes aluminum. In other embodiments, the metallic substrate 416 can be composed substantially of aluminum. In other embodiments, the metallic substrate 416 can be composed essentially of aluminum. In some embodiments, the metallic substrate 416 can include stainless steel, aluminum, or combinations thereof.

In some embodiments, the coating 418 can be a conductive polymer. In some embodiments, the conductive polymer includes polypyrrole. In other embodiments, the conductive polymer can be composed substantially of polypyrrole. In other embodiments, the conductive polymer can be composed essentially of polypyrrole. In some embodiments, the conductive polymer includes poly (3,4-ethylenedioxythiophene) polystyrene sulfonate. In some embodiments, the conductive polymer includes polypyrrole, poly (3,4-ethylenedioxythiophene) polystyrene sulfonate, or any combinations thereof.

In some embodiments, the conductive polymer includes a doping agent. In some embodiments, the polypyrrole can be doped with sodium dodecyl sulfate. In some embodiments, the polypyrrole can be doped with graphene. In some embodiments, the polypyrrole can be doped with sodium p-toluenesulfonate. In some embodiments, the polypyrrole can be doped with sodium dodecyl sulfate, graphene, sodium p-toluenesulfonate, or any combinations thereof.

In some embodiments, the coating 418 includes one or more additives. In some embodiments, the one or more additives are configured to improve a corrosion resistance, improve an electrical conductivity, or combinations thereof. For example, the coating 418 can include an additive for improving a corrosion resistance properties of the electrodes 400. In another example, the coating 418 can include an additive for improving an electrical conductivity of the electrodes 400.

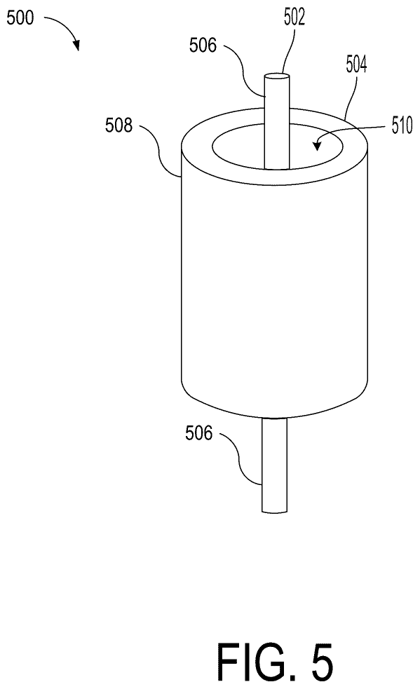

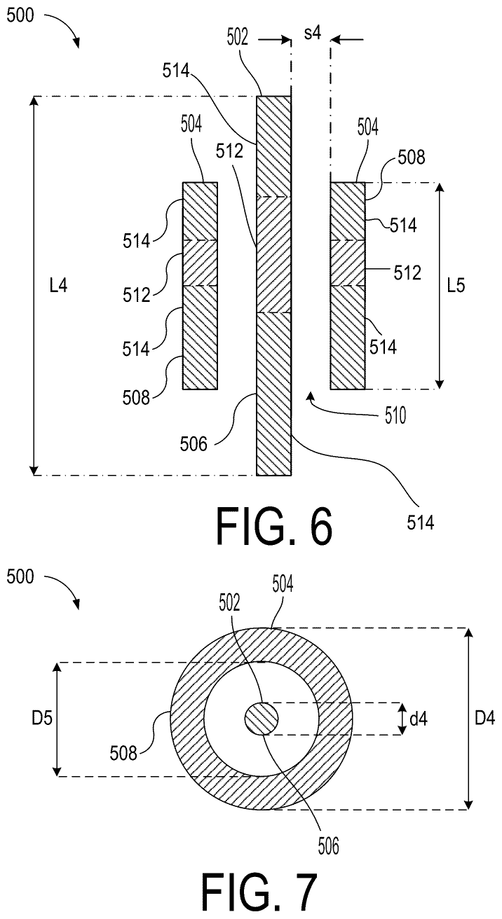

FIG. 5 is a perspective view of electrodes 500, according to some embodiments. FIG. 6 is a partially exposed side view of the electrodes 500 in FIG. 5, according to some embodiments. FIG. 7 is a top view of the electrodes 500 in FIG. 5, according to some embodiments. Unless stated otherwise, FIGS. 5-7 will be referenced collectively.

In some embodiments, the electrodes 500 can include electrode 502 and electrode 504. In some embodiments, the electrode 502 can be a cylinder 506 and the electrode 504 can be a cylinder 508. In some embodiments, at least a portion of the cylinder 506 can be disposed within an interior of the cylinder 508. That is, in some embodiments, the cylinder 508 can include a channel 510 extending therethrough, and the cylinder 506 can be arranged relative the cylinder 508 so as to extend through the channel 510 and so that at least a portion of the cylinder 506 is located in the channel 510. In some embodiments, the electrode 502 is a first cylinder and the electrode 504 is a second cylinder, the electrode 502 and the electrode 504 being colinear. In some embodiments, the electrode 502 is a first cylinder and the electrode 504 is a second cylinder, the electrode 502 and electrode 504 being coaxial so that at least a portion of the cylinder 506 is disposed within an interior of the cylinder 508.

Referring to FIG. 6, in some embodiments, the electrode 502 can have a length (L4). In some embodiments, the electrode 504 can have a length (L5). In some embodiments, the length (L4) of the electrode 502 can be greater than the length (L5) of the electrode 504.

In some embodiments, the cylinder 506 of electrode 502, or a portion thereof, can extend through an interior of the cylinder 508 of electrode 504. In some embodiments, the electrode 502 can be coaxial with the electrode 504. In some embodiments, the electrode 502 can be spaced a distance (s4) apart from the electrode 504. That is, in some embodiments, the distance between an outer surface of the electrode 502 and an inner surface of the channel 510 of electrode 504 can be the distance (s4).

In some embodiments, the electrode 502 and electrode 504 can be disposed in the fluid flow path between the fluid inlet and the fluid outlet of the conductivity sensor 112.

In some embodiments, the electrode 502 can include the metallic substrate 512. In some embodiments, the electrode 502 can also include the coating 514. In some embodiments, the coating 514 can cover at least a portion of the metallic substrate 512. In some embodiments, the coating 514 can cover the portion of the metallic substrate 512 that is in contact with the fluid in the fluid path. In other embodiments, the coating 514 can substantially cover the metallic substrate 512.

In some embodiments, the electrode 504 can include the metallic substrate 512. In some embodiments, the electrode 504 can also include the coating 514. In some embodiments, the coating 514 can cover at least a portion of the metallic substrate 512. In some embodiments, the coating 514 can cover the portion of the metallic substrate 512 that is in contact with the fluid in the fluid path. In other embodiments, the coating 514 can substantially cover the metallic substrate 512.

In some embodiments, the metallic substrate 512 includes stainless steel. In other embodiments, the metallic substrate 512 can be composed substantially of stainless steel. In other embodiments, the metallic substrate 512 can be composed essentially of stainless steel. In some embodiments, the metallic substrate 512 includes aluminum. In other embodiments, the metallic substrate 512 can be composed substantially of aluminum. In other embodiments, the metallic substrate 512 can be composed essentially of aluminum. In some embodiments, the metallic substrate 512 can include stainless steel, aluminum, or combinations thereof.

In some embodiments, the coating 514 can be a conductive polymer. In some embodiments, the conductive polymer includes polypyrrole. In other embodiments, the conductive polymer can be composed substantially of polypyrrole. In other embodiments, the conductive polymer can be composed essentially of polypyrrole. In some embodiments, the conductive polymer includes poly (3,4-ethylenedioxythiophene) polystyrene sulfonate. In some embodiments, the conductive polymer includes polypyrrole, poly (3,4-ethylenedioxythiophene) polystyrene sulfonate, or any combinations thereof.

In some embodiments, the conductive polymer includes a doping agent. In some embodiments, the polypyrrole can be doped with sodium dodecyl sulfate. In some embodiments, the polypyrrole can be doped with graphene. In some embodiments, the polypyrrole can be doped with sodium p-toluenesulfonate. In some embodiments, the polypyrrole can be doped with sodium dodecyl sulfate, graphene, sodium p-toluenesulfonate, or any combinations thereof.

In some embodiments, the coating 514 includes one or more additives. In some embodiments, the one or more additives are configured to improve a corrosion resistance, improve an electrical conductivity, or combinations thereof. For example, the coating 514 can include an additive for improving a corrosion resistance properties of the electrodes 500. In another example, the coating 514 can include an additive for improving an electrical conductivity of the electrodes 500.

Referring to FIG. 7, in some embodiments, the electrode 502 can have a diameter (d4). In some embodiments, the electrode 504 can have an outer diameter (D4). In some embodiments, the electrode 504 can have an inner diameter (D5).

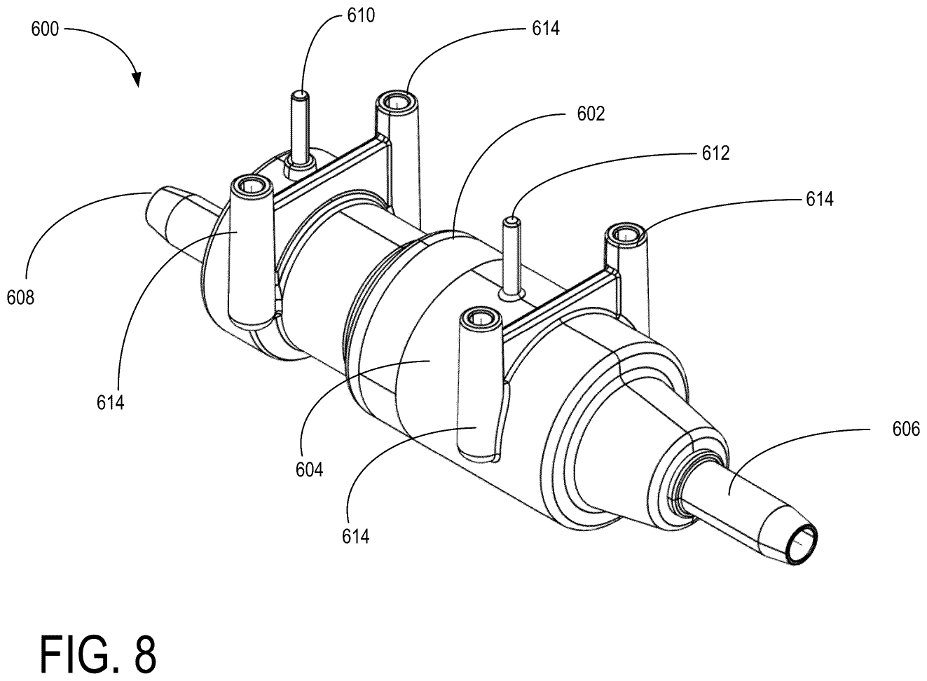

FIG. 8 is a perspective view of the conductivity sensor 600, according to some embodiments.

In some embodiments, dialysis system 100, as shown in FIG. 1, can include a water purification system 102. The water purification system 102 being configured to purify a fluid intended for dialysis. The water purification system 102 can include a conductivity sensor 112, according to some embodiments. In some embodiments, the dialysis system 100 can include conductivity sensor such as conductivity sensor 600. That is, in some embodiments, conductivity sensor 112 can be conductivity sensor 600. For example, the conductivity sensor 600 can be utilized in the water purification system 102 as shown in FIG. 1.

The conductivity sensor 600 can include a sensor body 602 defining a housing. In some embodiments, the sensor body 602 has at least one wall 604 enclosing a fluid flow path. The sensor body 602 has a fluid inlet 606 and a fluid outlet 608. The conductivity sensor 600 includes electrode 610 and electrode 612. The electrode 610 and electrode 612 are disposed in the fluid flow path between the fluid inlet 606 and the fluid outlet 608.

In some embodiments, the electrode 610 can be electrode 202 and electrode 612 can be electrode 204, as shown in FIG. 2. In some embodiments, the electrode 610 can be electrode 302 and electrode 612 can be electrode 304, as shown in FIG. 3. In some embodiments, the electrode 610 can be electrode 402 and electrode 612 can be electrode 404, as shown in FIG. 4. In some embodiments, the electrode 610 can be electrode 502 and electrode 612 can be electrode 504, as shown in FIG. 5.

A plurality of output connectors 614 also extend from the sensor body 602. As a result, the conductivity sensor 600 can be connected to a processor via the plurality of output connectors 614. That is, in some embodiments, the processor can be secured to the plurality of output connectors 614.

In some embodiments, an electrode connection can extend from the sensor body 602. In some embodiments, the electrode connection can extend from the sensor body 602, the electrode connection being in electrical connection with the electrode 610 within the sensor body 602. In some embodiments, an electrode can also extend from the sensor body 602. In some embodiments, the electrode connection can extend from the sensor body 602, the electrode connection being in electrical connection with the electrode 612 within the sensor body 602.

By utilizing the electrodes in the conductivity sensor such as, for example, conductivity sensor 112 and conductivity sensor 600, in-line contact conductivity measurements of high-impedance aqueous based solutions (e.g., fluids) can be performed. It is to be appreciated by those having ordinary skill in the art that the conductivity sensor and the electrodes (e.g., electrodes 114, electrodes 200, electrodes 300, electrodes 400, electrodes 500, electrode 610, and electrode 612), can include different geometry depending on the sensor application, sensor location, other factors, or any combinations thereof.

The one or more electrodes 114 in the conductivity sensor 112 have a metallic core that is formed of a lower cost metal relative to other known conductivity sensors that utilize higher cost metals such as, for example, silver, gold, platinum, and the like. The one or more electrodes 114 in the conductivity sensor 112 have a coating, the coating being characterized by a certain thickness. The coating can be made of a conductive polymer based on the chemical and electrical stability properties of the coating.

In some embodiments, the conductive polymer can be doped with one or more chemicals to increase the conductive properties of the one or more electrodes 114. For example, the coating can be made of a conductive polymer doped with sodium dodecyl sulfate (“SDS”) or sodium p-toluenesulfonate (“TsONa”). In some embodiments, the coating can be a mixture of a conductive polymer and can be doped with graphene to increase the conductive properties of the respective electrode 114 and to improve the resistance to coating penetration by corrosive elements such as, for example, in the fluid. For example, in some embodiments, the conductivity sensor 112 can include the electrodes 114, the electrodes 114 including a mixture of polypyrrole doped with sodium dodecyl sulfate and graphene.

In some embodiments, the coating disposed on the electrodes 114 can be produced by voltammetry electrodeposition. For example, in some embodiments, the electrodes including the coating can be formed using electrodeposition in a three-electrode configuration starting from a solution including 0.4 M of pyrrole, 0.15 M of SDS, and 50 g/L of graphene. In some embodiments, the polypyrrole can be synthesized during the electrodeposition by electro polymerization. In some embodiments, the solution may be treated with ultrasounds to increase the homogeneity before starting the electrodeposition process. In some embodiments, the coating thickness can be adjusted as a function of voltametric cycles performed during the electrodeposition process, and as a function of the voltage applied to the setup during the electrodeposition process.

### EXAMPLE

Several electrodes according to the present disclosure were tested. The components of the coating are shown below in Table 1.

TABLE 1
Sample Details

| | Pyrrole | SDS | TsONa |
|---|---|---|---|
| Sample | Concentration | Concentration | Concentration |
| # | (mol/L) | (mol/L) | (mol/L) |
| 1 | 0.5 | 0.03 | |
| 2 | 0.5 | 0.1 | |
| 3 | 0.5 | 0.15 | |
| 4 | 0.5 | 0.5 | |
| 5 | 0.5 | | 0.1 |
| 6 | 0.5 | | 0.35 |
| 7 | 0.5 | | 0.5 |

The samples of Table 1 were subjected to cyclic voltammetry having a potential range of 0-1.1 volts for 5 cycles at a scan rate of 50 mV/s. The results of the samples for various parameters are shown below in Table 2.

TABLE 2
Sample Conductivity Parameter Results

| | | | | | Charge |
|---|---|---|---|---|---|
| | Open | | | | Transfer |
| | Circuit | Corrosion | Corrosion | Polarization | Resistance |
| Sample | Potential | Velocity | Current | Resistance | at 2 kHz |
| # | (mV) | (μm/year) | (μA) | (kΩ) | (Ω) |
| 1 | 55 | 276 | 23.78 | 1.096 | 35 |
| 2 | | | | | 25 |
| 3 | 180 | 693 | 59.74 | 0.436 | 12 |
| 4 | 194 | 131 | 11.31 | 2.303 | 2 |
| 5 | | | | | 16 |
| 6 | | | | | 5 |
| 7 | 157 | 1,090 | 93.91 | 0.277 | 2 |

The results show that sample 4 can have preferable properties, including pyrrole and SDS, had a relatively high open circuit potential, low corrosion velocity and corrosion current, high polarization resistance, and low charge transfer resistance.

All prior patents and publications referenced herein are incorporated by reference in their entireties.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “poor” refers to a material's ability to be corrosion-resistant.

As used herein, the term “substantially parallel” refers to a direction of an axis relative a direction of another axis, the respective axis being in the same or similar direction.

As used herein, the term “composed substantially of” refers to a member that can be composed of one or more materials, and the member is formed to a significant extent of a certain material of the one or more materials.

As used herein, the term “impedance” refers to the opposition to alternating current in a medium due to a resistance, reactance, or both in the medium.

As used herein, the term “high-impedance” refers to an electrical resistance of greater than 500 k≠, resulting in an ultra-low electrical conductivity (e.g., 0.8-1.8 μS/cm).

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “between” does not necessarily require being disposed directly next to other elements. Generally, this term means a configuration where something is sandwiched by two or more other things. At the same time, the term “between” can describe something that is directly next to two opposing things. Accordingly, in any one or more of the embodiments disclosed herein, a particular structural component being disposed between two other structural elements can be:
disposed directly between both of the two other structural elements such that the particular structural component is in direct contact with both of the two other structural elements;
disposed directly next to only one of the two other structural elements such that the particular structural component is in direct contact with only one of the two other structural elements;
disposed indirectly next to only one of the two other structural elements such that the particular structural component is not in direct contact with only one of the two other structural elements, and there is another element which juxtaposes the particular structural component and the one of the two other structural elements;
disposed indirectly between both of the two other structural elements such that the particular structural component is not in direct contact with both of the two other structural elements, and other features can be disposed therebetween; or
any combination(s) thereof.

What is claimed is:

1. A dialysis system comprising:
a purification system configured to purify a fluid intended for dialysis; and
a conductivity sensor comprising:
a sensor body,
wherein the sensor body enclosing a fluid flow path;
wherein the sensor body has a fluid inlet and a fluid outlet;
a first electrode,
wherein the first electrode is disposed in the fluid flow path between the fluid inlet and the fluid outlet;
wherein the first electrode comprises:
a metallic substrate; and
a coating covering at least a portion of the metallic substrate; and
a second electrode,
wherein the second electrode is located in the fluid flow path between the fluid inlet and the fluid outlet.

2. The dialysis system of claim 1, wherein the coating comprises a conductive polymer.

3. The dialysis system of claim 2, wherein the conductive polymer comprises polypyrrole.

4. The dialysis system of claim 3, wherein the polypyrrole is doped with sodium dodecyl sulfate.

5. The dialysis system of claim 3, wherein the polypyrrole is doped with graphene.

6. The dialysis system of claim 3, wherein the polypyrrole is doped with sodium p-toluenesulfonate.

7. The dialysis system of claim 2, wherein the conductive polymer comprises poly (3,4-ethylenedioxythiophene) polystyrene sulfonate.

8. A conductivity sensor comprising:
a sensor body,
wherein the sensor body enclosing a fluid flow path;
wherein the sensor body has a fluid inlet and a fluid outlet;
a first electrode,
wherein the first electrode is disposed in the fluid flow path between the fluid inlet and the fluid outlet;
wherein the first electrode comprises:
a metallic substrate; and
a coating covering at least a portion of the metallic substrate; and
a second electrode,
wherein the second electrode is located in the fluid flow path between the fluid inlet and the fluid outlet.

9. The conductivity sensor of claim 8, wherein the metallic substrate comprises stainless steel.

10. The conductivity sensor of claim 8, wherein the metallic substrate comprises aluminum.

11. The conductivity sensor of claim 8, wherein the coating comprises a conductive polymer.

12. The conductivity sensor of claim 11, wherein the conductive polymer comprises polypyrrole.

13. The conductivity sensor of claim 12, wherein the polypyrrole is doped with sodium dodecyl sulfate.

14. The conductivity sensor of claim 12, wherein the polypyrrole is doped with sodium p-toluenesulfonate.

15. The conductivity sensor of claim 12, wherein the polypyrrole is doped with graphene.

16. The conductivity sensor of claim 11, wherein the conductive polymer comprises poly (3,4-ethylenedioxythiophene) polystyrene sulfonate.

17. The conductivity sensor of claim 11, wherein the coating comprises one or more additives.

18. The conductivity sensor of claim 17, wherein the one or more additives are configured to improve a corrosion resistance, improve an electrical conductivity, or combinations thereof.

19. The conductivity sensor of claim 11, wherein the first electrode is a first cylinder and the second electrode is a second cylinder, wherein the first cylinder and the second cylinder are disposed so that longitudinal axes thereof are parallel.

20. A conductivity sensor comprising:
a sensor body,
wherein the sensor body enclosing a fluid flow path;
wherein the sensor body has a fluid inlet and a fluid outlet;
a first electrode,
wherein the first electrode is disposed in the fluid flow path between the fluid inlet and the fluid outlet;
wherein the first electrode comprises:
a metallic substrate; and
a coating covering at least a portion of the metallic substrate; and
a second electrode,
wherein the second electrode is located in the fluid flow path between the fluid inlet and the fluid outlet,
wherein the first electrode is a first plate and the second electrode is a second plate, wherein the first plate and the second plate are disposed so that major surfaces thereof are parallel.
